(19) United States
(12) Patent Application Publication
(10) Pub. No.: US 2017/0001052 A1
(43) Pub. Date: Jan. 5, 2017

(54) METHOD FOR ACTIVATING OXYGENASE-CONTAINING COMPOSITION AND CONTAMINANT DETOXIFICATION METHOD AND DEVICE BASED ON SAME

(71) Applicants: TAKAHATA ELECTRONICS CORPORATION, Yonezawa-shi, Yamagata (JP); NATIONAL UNIVERSITY CORPORATION YAMAGATA UNIVERSITY, Yamagata-shi, Yamagata (JP); APPLIZYME INC., Yonezawa-shi, Yamagata (JP)

(72) Inventors: Tomijiro Hara, Yonezawa-shi (JP); Yumiko Takatsuka, Yonezawa-shi (JP); Sonoka Katakura, Yonezawa-shi (JP); Toshinori Makuta, Yonezawa-shi (JP); Katsunori Sano, Yonezawa-shi (JP); Tokio Niikuni, Yonezawa-shi (JP)

(73) Assignees: TAKAHATA ELECTRONICS CORPORATION, Yonezawa-shi, Yamagata (JP); NATIONAL UNIVERSITY CORPORATION YAMAGATA UNIVERSITY, Yamagata-shi, Yamagata (JP); APPLIZYME INC., Yonezawa-shi, Yamagata (JP)

(21) Appl. No.: 14/902,533

(22) PCT Filed: Jul. 4, 2014
(86) PCT No.: PCT/JP2014/067939

§ 371 Date: Dec. 31, 2015

(30) Foreign Application Priority Data
Jul. 5, 2013 (JP) 2013-141383

(51) Int. Cl. A62D 3/02; C12M 1/40; C12M 1/06; C12N 9/02

(52) U.S. Cl. CPC A62D 3/02; C12N 9/0071; C12Y 114/12018; C12M 21/18; C12M 27/02; A62D 2101/22

(57) ABSTRACT

Provided is a method for activating an oxygenase-containing composition, characterized by dissolving or dispersing a composition containing oxygenase in an aqueous medium containing oxygen in an amount that exceeds the saturated dissolved oxygen concentration in an atmospheric environment at normal temperature and normal pressure.

### TECHNICAL FIELD

The present invention relates to a method for decomposing or detoxifying contaminants including monocyclic aromatic compounds such as toluene or benzene and polycyclic aromatic hydrocarbons such as dioxins or polychlorinated biphenyls, and to a device used for that purpose. More particularly, the present invention relates to a method for decomposing a contaminant by an oxidation-reduction reaction by aerating and/or stirring a mixture of an oxygenase-containing composition and an oily component containing the aforementioned contaminant in an aqueous medium having an elevated dissolved oxygen concentration by dispersing microbubbles therein, and to a device used for that purpose.

### BACKGROUND ART

Polychlorinated biphenyls (PCBs) are an example of a contaminant that constitute a type of poorly biodegradable organic chlorine compounds, and although these compounds had been widely used throughout industry due to their chemically stable properties, ever since they were determined to accumulate in the body and demonstrate disrupting effects on the endocrine system, their production and use have been prohibited on an international scale. Since PCBs are chemically stable and remain for a long period of time without decomposing spontaneously, they present the serious problem of having an effect not only on humans, but also on various other living organisms residing on the earth.

Approximately 55,000 tons of PCBs are estimated to have been imported, manufactured and sold in Japan in the past. After their use has been prohibited and users have been subjected to mandatory storage, a plan has been drafted that calls for detoxification by a certain time period in accordance with laws such as the Special Measures Law relating to promoting the proper disposition of polychlorinated biphenyl waste products. Although it has recently emerged that there are numerous cases of the detection of trace amounts of PCBs on the order of several tens of mg per kg in insulating oil used in electrical equipment, accurate quantities relating to usage or storage of this contaminated oil containing trace amounts of PCBs have yet to be ascertained. Known examples of methods used to decompose PCBs include conventional incineration methods as well as dechlorination decomposition, hydrothermal oxidative decomposition, reduction thermochemical decomposition using a hydrogen donor, and photodecomposition by UV irradiation. Among these, ultraviolet decomposition consists of dissolving PCBs in a polar organic solvent and irradiating with ultraviolet light to dechlorinate the solution and detoxify the residual PCBs by biological treatment or catalytic treatment and the like, and since this method offers a high degree of safety since treatment can be carried out at normal temperature and normal pressure while the decomposition products thereof are also presumed to be highly safe since toxic PCBs are catabolized by living organisms in the form of microorganisms, it is thought to be advantageous in comparison with chemical treatment and the like.

For example, the method described in Patent Document 1 consists of first carrying out dechlorination treatment by exposing PCBs to ultraviolet light followed by decomposing with microorganisms in a large-scale fermentation plant. However, this treatment method has the problem of treating a large volume of oil contaminated with a high concentration of PCBs reaching as much as 60% (w/v) to 80% (w/v) all at once, thereby making it necessary to adjust PCB concentration by adding a large amount of medium in the microbial treatment step following the ultraviolet irradiation step, and resulting in the difficulty of having to carry out microbial culturing and growth simultaneous to PCB decomposition.

Meanwhile, there has recently been a growing trend towards the use of gas bubbles having a diameter of 100 μm or less in the form of microbubbles in not only industrial applications, but also in the agricultural and marine products industries and medicine. Since microbubbles have a large surface area per volume and an extremely slow ascent rate, they enable a gas such as oxygen to be effectively dissolved in a liquid. In addition, they uniformly disperse in a liquid as a result of taking on an electrical charge. For example, filling a water purification tank with oxygen microbubbles in an aeration process is reportedly effective for activating and improving the efficiency of aerobic microorganisms present in activated sludge (see Non-Patent Document 1). In addition, although a method is reported in Patent Document 2 that consists of purifying contaminated soil or groundwater contaminated with volatile organic compounds using microbubbles and microorganisms that thrive in the aforementioned contaminated soil, a process is not known wherein an enzymatic reaction per se that decomposes a contaminant is promoted by microbubbles.

### PRIOR ART DOCUMENTS

### Patent Documents

Patent Document 1: Japanese Unexamined Patent Publication No. 2001-46547
Patent Document 2: Japanese Unexamined Patent Publication No. 2012-40476

### Non-Patent Documents

Non-Patent Document 1: Terasaka, K. et al., Chem. Eng. Sci., 2011, Vol. 66, pp. 3172-3179

### DISCLOSURE OF THE INVENTION

### Problems to be Solved by the Invention

An object of the present invention is to provide a means for improving the efficiency of a process for decomposing contaminants derived from monocyclic aromatic compounds such as toluene or benzene and polycyclic aromatic hydrocarbons such as dioxins and polychlorinated biphenyls by an oxidation-reduction reaction using an enzyme. In particular, an object of the present invention is to provide a method and device for efficiently decomposing and detoxifying these contaminants by promoting a reaction by oxygenase that plays an important role in promoting a series of decomposition reactions for the initial oxidation reaction.

### Means for Solving the Problems

According to an analysis conducted by the inventors of the present invention, in a conventional fermentation method in which the production of a decomposing enzyme by aerobic microorganisms and decomposition of a contaminant are carried out simultaneously, it is difficult to supply an adequate amount of oxygen required by this fermentation reaction in the case of a culture broth under a standard oxygen partial pressure in the atmosphere. In contrast, it was found that, by using an oxygenase composition preliminarily produced using microorganisms and the like, such as a microbial preparation in which multicomponent enzymes, including an aromatic ring-hydroxylating dioxygenase, are expressed in large amounts in microbial cells, while also enhancing the dissolved oxygen concentration in an aqueous medium by dispersing microbubbles in the aqueous medium in which the enzymatic reaction proceeds, the supply of molecular oxygen used to decompose a substrate is enhanced, each component involved in the decomposition reaction is adequately dispersed enabling them to efficiently make contact, and as a result thereof, the decomposition rate of contaminants improves remarkably. The present invention was completed on the basis of these findings.

Namely, in one aspect of the present invention, the present invention provides a method for activating an oxygenase-containing composition, comprising dissolving or dispersing a composition containing oxygenase in an aqueous medium containing an amount of oxygen that exceeds the saturated dissolved oxygen concentration in an atmospheric environment at normal pressure. In a different aspect, the present invention provides a method for decomposing or detoxifying a contaminant, wherein a mixture of an oxygenase-containing composition and an oily component containing a contaminant is stirred in an aqueous medium containing an amount of oxygen that exceeds the saturated dissolved oxygen concentration. At least a portion of the oxygen in the aforementioned aqueous medium is preferably present in the form of microbubbles, and thus, one embodiment of the method of the present invention includes the providing of microbubbles to the aforementioned aqueous medium.

In another aspect of the present invention, the present invention provides a device that can be used in the aforementioned method for decomposing or detoxifying a contaminant, wherein the device is provided with a stirring tank provided with an aeration means, stirring means and/or temperature control means, a sample introduction unit in communication with the aforementioned stirring tank that introduces an aqueous medium and/or oily component containing a contaminant, and a microbubble generation unit that supplies microbubbles to a sample in the aforementioned stirring tank. The microbubble generation unit is preferably an ultrasonic microbubble generation device provided in the aforementioned stirring tank or a pressurized microbubble generation device provided in the aforementioned sample introduction unit.

### Effects of the Invention

According to the method of the present invention, the reaction rate of oxygenase is improved by enhancing the supply of molecular oxygen used to decompose contaminants. Moreover, improvement of decomposition efficiency makes it possible to efficiently detoxify contaminants even at comparatively low concentrations. In addition, an aqueous medium containing microbubbles improves the dispersibility of an enzyme preparation such as a microbial preparation, an emulsion with an oily component containing contaminants is formed easily, and an environment is thought to be created that is suitable for decomposition of contaminants.

In addition, since the contaminant detoxification device of the present invention is able to efficiently supply microbubbles to an aqueous medium, while also being able to supply oxygen in microbubbles at suitable times for the purpose of supplementing the dissolved oxygen concentration that begins to decrease as the decomposition reaction proceeds, contaminants are thought to be able to be detoxified more efficiently.

### BRIEF DESCRIPTION OF THE DRAWINGS

FIG. 1 shows the results of electrophoresis carried out in Reference Example 2 indicating the presence of a composition that expresses an oxygenase gene.

FIG. 2 shows the results of a total ion chromatogram output by a gas chromatograph mass spectrometer indicating that a composition expressing an oxygenase gene decomposes PCBs as carried out in Example 1.

FIG. 3 is a schematic cross-sectional view of a contaminant detoxification device according to one embodiment of the present invention.

FIG. 4 is a perspective view showing a schematic representation of a microbubble generation device preferably used in the method or detoxification device of the present invention.

FIG. 5 shows the results of a PCB decomposition assay carried out in Example 2.

FIG. 6 shows the results of a time-based PCB decomposition assay carried out in Example 3.

FIG. 7 shows the structure of a biphenyl dioxygenase complex expression plasmid pEA1A2A3A4 (LB400) derived from Burkholderia xenovorans strain LB400.

FIG. 8 is a graph showing time-based changes in the amount of residual PCBs when a PCB-contaminated insulating oil was decomposed using a PCB decomposition device in Example 4.

### BEST MODE FOR CARRYING OUT THE INVENTION

The following respectively provides detailed descriptions of the method for activating an oxygenase-containing composition according to the present invention, a method for detoxifying contaminants based thereon, and a detoxification device able to be used therein. In the present description, the term “detoxification” means lowering toxicity with respect to living organisms, and refers to lowering to a concentration so as to be able to reduce detrimental effects on living organisms even in the case of having been released into the environment, although not necessarily requiring that a contaminant be completely decomposed, and preferably refers to lowering to a concentration that is generally recognized to be safe for each substance or below a reference value established by laws and the like. The contaminant detoxification method of the present invention is characterized by mixing an oxygenase-containing composition with an oily component containing a contaminant in an aqueous medium dispersed with highly concentrated oxygen and microbubbles, and decomposing the mixture by aeration and/or stirring, each constituent of which is explained in order below.

[Oxygenase-Containing Composition]

There are no particular limitations on the oxygenase-containing composition able to be used in the method of the present invention provided it is an enzyme capable of oxidatively decomposing a contaminant, and numerous oxygenases derived from animals or microorganisms can be used to decompose various types of contaminants. Among these, a group of enzymes referred to as aromatic ring-hydroxylating dioxygenases or Rieske non-heme iron oxygenases is preferable, and these enzymes catalyze the initial reaction of numerous aromatic compound decomposition pathways in the form of a cis-type dihydroxylation reaction. These enzymes play an important role as the initial oxidase that influences whether or not a series of decomposition reactions is promoted in aerobic metabolic pathways of various aromatic compounds, including not only monocyclic and polycyclic aromatic hydrocarbons such as toluene and naphthalene as well as benzene, cumene, phenanthrolene and pyrene, but also heterocyclic aromatic compounds such as dioxins, dibenzothiophene or carbazole, as well as biphenyl ring compounds such as PCBs. Aromatic ring-hydroxylating dioxygenases are multicomponent enzymes composed of a terminal oxidase (TO), which recognizes a substrate and carries out an oxidation reaction, and an electron transport system that transfers electrons to TO from NAD(P)H. The electron transport system may be composed of a reductase (Red) alone that accepts electrons from NAD(P)H, or may be composed of two components consisting of Red and ferredoxin (Fdx).

Microorganisms are believed to have developed various types of aromatic ring-hydroxylating dioxygenases through exposure to aromatic compounds, adaptation thereto and evolution, and at present, more than 200 aromatic ring-hydroxylating dioxygenases have been isolated as enzymes decomposing more than several tens of compounds. Classifications such as classification of aromatic ring-hydroxylating dioxygenases according to characteristics of the electron transport chain and classification according to the molecular evolutionary tree of each enzyme based on the amino acid sequence alignment of oxidase catalytic subunits are described in “Nojiri, H., et al., Protein, Nucleic Acid and Enzyme, Vol. 50, No. 12 (2005), pp. 1519-1526”, and the entire content thereof is incorporated in the present application by reference.

In a preferred embodiment, the oxygenase-containing composition used in the method of the present invention is a microbial preparation obtained by expressing the aforementioned aromatic ring-hydroxylating dioxygenase in microbial cells, and contains other metabolic enzymes capable of further decomposing intermediate products formed by a dihydroxylation reaction and ultimately converting to energy substances such as acetyl CoA or pyruvic acid. Examples of the series of enzyme groups involved in biphenyl decomposition, in addition to an aromatic ring-hydroxylating dioxygenase in the form of biphenyl dioxygenase (BphA enzyme), include dihydrodiol dehydrogenase (BphB enzyme), which removes two hydrogen atoms from the reaction product of BphA; 2,3-dihydroxybiphenyl dioxygenase (BphC enzyme), which forms 2-hydroxy-6-oxo-6-phenylhexa-2,4-dienoic acid (HOPDA) by adding a molecule of oxygen to the reaction product of BphB; and 2-hydroxy-6-oxo-6-phenylhexa-2,4-dienoate hydrolase (BphD enzyme), which decomposes HOPDA into benzoic acid and 2-hydroxypenta-2,4-dienoic acid; as well as 2-hydroxypenta-2,4-dienoate hydratase (BphE enzyme), which converts to acetyl CoA and pyruvic acid; 4-hydroxy-2-oxovalerate aldolase (BphF enzyme); and acetaldehyde dehydrogenase (BphG enzyme). The material conversion pathways and the like used by this series of enzyme groups involved in biphenyl decomposition are described in “Hara, T. and Takatsuka, Y., Industry and the Environment, October 2010, pp. 65-69”, and the entire content thereof is incorporated in the present application by reference.

These microorganisms having aromatic ring-hydroxylating dioxygenase can be screened by adding a target contaminant present in the natural environment or a compound having a similar structure thereto to media according to methods known among persons with ordinary skill in the art. For example, microorganisms that decompose PCBs can be found by repeatedly screening from among microorganisms capable of growing using biphenyls as their only carbon source. Although examples of microorganisms that produce biphenyl-decomposing enzymes in nature include numerous bacteria belonging to the genii Pseudomonas, Comamonas, Burkholderia, Sphingomonas, Rhodococcus and Ralstonia, microorganisms demonstrating superior PCB decomposition activity can be expected to be acquired by selecting microorganisms that grow rapidly and rapidly produce meta-cleaving substances (such as 2-hydroxy-6-oxo-6-phenylhexa-2,4-dienoate), which exhibit a yellow-orange color to serve as an indicator of biphenyl decomposition activity, in the screening stage of biphenyl-assimilating microorganisms.

Moreover, gene clusters having further improved PCB decomposition activity can be created by cloning an enzyme gene involved in decomposition of a substance, such as a biphenyl-decomposing enzyme gene, from microorganisms obtained by screening, and introducing mutations therein by site-specific mutagenesis and the like. Furthermore, the Kunkel method, gapped duplex method and other known techniques or methods equivalent thereto can be used to introduce a mutation into a gene. In addition, gene mutagenesis and construction of chimeric genes can be carried out by techniques such as error-prone PCR or DNA shuffling. Error-prone PCR and DNA shuffling are techniques known in the art, and for example, error-prone PCR is described in Chen, K. and Arnold, F. H., 1993, Proc. Natl. Acad. Sci. USA, 90, 5618-5622, while molecular evolution engineering techniques such as DNA shuffling and cassette PCR are described in Kurtzman, A. L., Govindarajan, S., Vahle, K., Jones, J. T., Heinrichs, V. and Patten, P. A., Advances in directed protein evolution by recursive genetic recombination: Applications to therapeutic proteins, Curr. Opinion Biotechnol., 12, 361-370, 2001. Mutant genes created using these techniques can be used to create novel microorganisms by either substituting with genomic DNA of the original microorganisms, or by cloning in plasmid DNA or cosmid DNA and introducing it into a host microorganism. Microorganisms that produce oxygenase capable of being used in the method of the present invention, and preferably aromatic ring-hydroxylating dioxygenase, are considered to be able to be easily acquired by such methods by a person with ordinary skill in the art.

There are also no particular limitations on the method used to culture microorganisms producing oxygenase, and oxygenase can be extracted and purified in accordance with ordinary methods from microbial cells obtained by culturing using culturing methods and culturing devices known among persons with ordinary skill in the art.

Alternatively, a culture containing microorganisms that produce oxygenase can be used directly in the form of intracellular enzyme by either crushing as is or washing with water or a dispersion medium containing a surfactant and the like. A culture broth obtained following culturing can be used as is or concentrated under reduced pressure. In addition, microorganisms producing a high concentration of oxygenase can be isolated and recovered by carrying out harvesting by centrifugal separation, density gradient centrifugation or biphasic separation. A suspension may also be used in which microorganisms producing oxygenase have been dispersed in various types of dispersion media.

When producing a liquid composition, substances can be added to the aforementioned culture as necessary for the purpose of improving storageability and stability. Examples of substances that can be added include pH adjusters, preservatives, antioxidants, stabilizers and buffers.

In the case of a powder composition, it is necessary to dry microbial cells obtained from the aforementioned culture. Microbial cells can be dried by powdering while remaining viable, by air drying, freeze-drying or spray drying and the like. At this time, it is preferable to use a protective agent such as skim milk. In addition, any substance such as an extender can also be added for formulation. Examples of excipients include sugars such as D-mannitol, D-sorbitol or sucrose, starches such as cornstarch or potato starch, inorganic salts such as calcium phosphate, calcium sulfate or precipitated calcium carbonate, as well as any excipient approved in the Feed Safety Law, such as defatted rice bran, soybean flour, bean curd refuse, peanut skin, bran, rice hull flour, calcium carbonate, sugar, starch, brewer's yeast or wheat flour. One type of these excipients may be used alone or two or more types may be used in combination.

In a preferred embodiment of the present invention, microbial cells are preferably washed at least twice with physiological saline or 20 mM phosphate buffer, and sodium phosphate is preferably used for the phosphate. In addition, an excipient such as a sugar-alcohol that may be alpha-, beta- or delta-mannitol may be added to the microbial cells so that they can be ultimately stored in a freezer and the like controlled to a temperature of −20° C. to −80° C., and in the case of drying to a powder, an oxygenase-containing composition is obtained that can be stored at a normal temperature of 15° C. to 25° C.

In addition, the oxygenase composition is preferably a composition obtained by compounding at a suitable blending ratio so as to efficiently decompose a contaminant, and for example, may be an oxygenase composition that is composed of a microbial preparation obtained by compounding microbial cells in the state of wet microbial cells prior to drying at a suitable blending ratio so as to efficiently decompose a contaminant, and then adding an excipient such as a sugar-alcohol to this complex.

For example, in order to decompose contaminants in the form of PCBs, at least one species or more of PCB-decomposing microorganisms can be used that are selected from the group consisting of Comamonas sp., Pseudomonas sp., Achromobacter sp., Rhodococcus sp. and Stenotrophomonas sp. These microbial cells demonstrate substrate specificity commonly selected by 2,3-biphenyl dioxygenase, and demonstrate an even narrower range of substrate specificity with respect to PCB isomers. More specifically, a plurality of bacteria that selectively decompose 2,2′,3,4-tetrachlorobiphenyl, 3,3′,4,4′-tetrachlorobiphenyl, 2,3,3′,6-tetrachlorobiphenyl, 3,4,4′-trichlorobiphenyl, 2,2′,3,4′-tetrachlorobiphenyl, 2,3,3′,4′-tetrachlorobiphenyl, 2,3,4,4′-tetrachlorobiphenyl, 2,2′,3,5,5′-pentachlorobiphenyl, 2,2′,4,4′-tetrachlorobiphenyl, 2,2′,4,5-tetrachlorobiphenyl and 2,2′,3,5′-tetrachlorobiphenyl are preferably mixed and compounded.

[Method for Activating Oxygenase-Containing Composition]

An oxygenase-containing composition can be activated by dissolving or dispersing such an oxygenase-containing composition in an aqueous medium containing oxygen in an amount that exceeds the saturated dissolved oxygen concentration in an atmospheric environment at normal temperature and normal pressure. In particular, remarkable activating effects can be obtained in the case of using the microbial preparation described above for the oxygenase-containing composition. Although the mechanism is currently not clear as to how the oxygenase-containing composition is activated by an increase in dissolved oxygen concentration in the aqueous medium, that is, how, for example, the substrate-decomposing reaction is improved by a microbial preparation in which aromatic ring-hydroxylating dioxygenase has been highly expressed in advance, one possible factor is that, as a result of an increase first in oxygen concentration in the aqueous medium outside the microbial cells, the oxygen concentration within the microbial cells rapidly increases to a level close to the oxygen concentration in the aqueous medium in an extremely short period of time due to passive transport. Next, an equilibrating phenomenon between the intracellular and extracellular oxygen concentrations of the microbial cells contributes to improving the speed of the oxygenation reaction on the substrate of the aromatic ring-hydroxylating dioxygenase that is carried out within the microbial cells. Moreover, the product of this oxygenation reaction is thought to undergo material conversions to smaller molecules by other metabolic enzymes and coenzymes contained in the enzyme composition that has been activated by oxidative stress induced at a high oxygen concentration, thereby allowing complete decomposition of the substrate.

[Microbubble-Containing Aqueous Medium]

In the present invention, the term “microbubble” refers to a gas bubble having a diameter of roughly 1 mm or less and preferably a diameter of 100 μm or less. Although gas bubbles may be formed by supplying a gas such as oxygen or air from the outside, or oxygen or air and the like dissolved in an aqueous medium may be used, in order to enhance the dissolved oxygen concentration in the aqueous medium, microbubbles are preferably generated while supplying oxygen gas from the outside. Since microbubbles have a large surface area per volume and an extremely slow ascent rate, they allow a gas such as oxygen to be effectively dissolved in a liquid. In addition, microbubbles uniformly disperse in a liquid as a result of being charged, and promote emulsification of an oily component in the aqueous medium. Since microbubbles have a negative surface charge, they can be uniformly dispersed in the aqueous medium through interaction with microbial cells and the like typically having a positive surface charge.

The step for dispersing microbubbles in the aqueous medium may be carried out before mixing with an oily component containing a contaminant, or the aqueous medium may be mixed with an oily component containing a contaminant followed by generating microbubbles in the mixture. Examples of methods for generating microbubbles include a method consisting of jetting gas into a liquid through a porous body or pipe having micropores, a method consisting of engulfing a gaseous phase in a liquid phase by utilizing shear force generated in a jet flow or rotational flow, and a method consisting of generating fine gas bubbles by vibrating a gas-liquid interface using ultrasonic waves, and any of these methods may be used.

In the method of the present invention, an amount of oxygen that at least exceeds the saturated dissolved oxygen concentration in the aqueous medium is preferably contained in the aqueous medium, and microbubbles are preferably generated by carrying out ultrasonic treatment while allowing oxygen gas to flow in for this purpose. These microbubbles are subsequently referred to as oxygen microbubbles. Although the saturated dissolved oxygen concentration in a solution varies according to such factors as air pressure, water temperature or dissolved salt concentration, the dissolved oxygen concentration of distilled water at 30° C. under atmospheric pressure is about 7.5 mg/L. In the method of the present invention, the dissolved oxygen concentration in the aqueous medium at 30° C. is at least an initial concentration of about 8 mg/L, preferably 15 mg/L or more and more preferably 25 mg/L (ppm) or more. In one embodiment, in the case of having filled the aqueous medium with oxygen microbubbles by the aforementioned ultrasonic generation method, the dissolved oxygen concentration thereof in terms of the actual measured value is about 28 mg/L. In general, oxygen in a highly concentrated state that has been dissolved in an aqueous medium is considered to demonstrate a decrease in dissolved oxygen concentration due to the characteristic of attempting to maintain equilibrium with the oxygen concentration in the surrounding environment. Thus, in order to optimize the decomposition reaction of a contaminant by the oxygenase-containing composition, the dissolved oxygen concentration that has been elevated to about 28 mg/L is preferably maintained and it is preferable to continue to suitably supply oxygen microbubbles either continuously or intermittently from a microbubble generator.

[Contaminant Decomposition Reaction]

Although there are no particular limitations on contaminants targeted by the decomposition or detoxification method of the present invention provided they are oxidatively decomposed by oxygenase, monocyclic or polycyclic aromatic compounds are preferable, and these include toluene and/or benzene or dioxins and/or polychlorinated biphenyls. In the present description, dioxins is the generic term for all polychlorinated dibenzo-p-dioxins, polychlorinated dibenzofurans and coplanar PCBs (particularly polychlorinated biphenyls in which chlorine atoms are substituted at positions other than the ortho position). In the present invention, “dioxins” represent all or a portion of these compounds unless specifically indicated otherwise.

Examples of PCBs targeted by the present invention include compounds in which chlorine atoms are substituted in a biphenyl compound, and the number of these substituted chlorine atoms is 1 to 10. The average number of substituted chlorine atoms is typically 2 to 6. In the present invention, at least one type of compound selected from these PCBs can be used, and one type can be used alone or two or more types can be arbitrarily used in combination. In general, PCBs are not present as single compounds, but rather exist in the form of compounds having different numbers of chlorine atoms and different substitution sites. Thus, there are theoretically 209 types of isomers based on the possible combinations of numbers of chlorine atoms and substitution sites, and roughly 70 to 100 or possibly more isomers are incorporated and present in commercially available products.

Characteristic examples of PCBs able to be treated by the decomposition or detoxification method of the present invention include, but are not limited to, 3,4,4′,5-tetrachlorobiphenyl, 3,3′,4,4′-tetrachlorobiphenyl, 3,3′,4,4′,5-pentachlorobiphenyl, 2,3,3′,4,4′-pentachlorobiphenyl, 2,3,4,4′,5-pentachlorobiphenyl, 2,3′,4,4′,5-pentachlorodiphenyl and 2′,3,4,4′,5-pentachlorobiphenyl, as well as 2,2′,4,4′-tetrachlorobiphenyl, 2,2′,4,5-tetrachlorobiphenyl and 2,2′,3,5′-tetrachlorobiphenyl.

PCBs had previously been commercially available as compounds composed of individual PCBs, and were used as insulating oil for capacitors and transformers. Even at present, a portion of these are contained in some capacitors and transformers in the form of PCBs having comparatively low concentrations as a result of being diluted with insulating oil. Specific examples thereof include Kanechlor KC-200 (in which the contained isomers primarily consist of dichlorinated phenyls), KC-300 (in which the contained isomers primarily consist of trichlorinated phenyls), KC-400 (in which the contained isomers primarily consist of tetrachlorinated biphenyls), KC-500 (in which the contained isomers primarily consist of pentachlorinated biphenyls), KC-600 (in which the contained isomers primarily consist of hexachlorinated biphenyls) and KC-1000 (mixture of KC500 and trichlorobenzene at a ratio (weight ratio) of 60/40), which were manufactured and sold by Kanegafuchi Chemical Ind. Co., Ltd., and Arochlor 1254 (54% chlorine), which was manufactured and sold by Mitsubishi Monsanto Chemical Co.

The method for decomposing or detoxifying contaminants in one embodiment of the present invention comprises a step for mixing the aforementioned oxygenase-containing composition and an oily component containing a contaminant in a highly concentrated oxygen microbubble-containing aqueous medium, and a step for aerating and stirring the mixture. In a preferred embodiment of the present invention, when using PCB-contaminated oil having a comparatively low contaminant concentration, high PCB decomposition activity can be demonstrated and the oil can be effectively detoxified.

In another embodiment, a contaminant may be decomposed by stirring and contacting the aforementioned oxygenase-containing composition and an oily component containing a contaminant in an aqueous medium while further aerating and supplying oxygen microbubbles to the mixture and dispersing therein. Moreover, by combining these embodiments, in addition to dispersing and contacting the aforementioned oxygenase-containing composition and oily component containing a contaminant in an aqueous medium preliminarily dispersed with oxygen microbubbles, microbubbles can be additionally supplied so as to supplement decreases in dissolved oxygen concentration in the mixture while stirring.

According to still another embodiment, in the case of using a microbial preparation as an oxygenase composition, the microbial preparation can be crushed by subjecting the mixture to ultrasonic treatment at a prescribed time during the decomposition step to release and disperse enzymes within the microbial cells in the mixture. Although aromatic compounds, including dioxins and PCBs, are dissolved to saturation or dispersed in an oily component since they are typically hydrophobic, they are believed to pass the cell membranes when contacting microbial cells and be subjected to decomposition reactions by a series of oxygenases present in the bacterial cells. However, when mass transfer within the mixture becomes the rate-determining step of the decomposition reactions due to a decrease in contaminant concentration as the decomposition reactions progress, the enzymes are preferably released into the mixture from bacterial cells by ultrasonic treatment and allowed to react with residual contaminants.

In one embodiment, the aforementioned mixture may form an emulsion with the aqueous medium and oily component, and this may be an oil-in-water (o/w) emulsion or water-in-oil (w/o) emulsion. Although the mixing ratio between the aforementioned aqueous medium and oily component may be an arbitrary ratio from 3:7 to 100:1, the ratio is preferably 3:7 to 7:3, more preferably 1:2 to 2:1, and most preferably about 1:1 in order to form an emulsion. The contaminant to be decomposed can be contained in the emulsion at about 0.05 mg/L to 1000 mg/L, and preferably at about 1 mg/L to 100 mg/L, based on the total quantity of the emulsion, and the decomposition reaction can be carried out by adding the oxygenase-containing composition at about 0.2% by weight to 20% by weight, and preferably about 2% by weight to 12% by weight, to the emulsion. In the case of not emulsifying, a surfactant such as Triton X-100 is added at 0.005% and the mixture is further homogenized by applying ultrasonic waves when necessary. Moreover, treatment for lowering the viscosity of the oily component, such as the addition of an alcohol and the like, may be carried out to further promote emulsification.

Surfactants able to be used in a preferred embodiment of the present invention are classified into nonionic, anionic, cationic and amphoteric surfactants and the like. Examples of nonionic surfactants include polyoxyethylene sorbitan monooleate (specifically, Polysorbate 80), polyoxyethylene polyoxypropylene glycol (specifically, Pluronic F68), sorbitan fatty acids (specifically, sorbitan monolaurate and sorbitan monooleate), polyoxyethylene derivatives (specifically, polyoxyethylene hydrogenated castor oil 60 and polyoxyethylene lauryl alcohol), glycerin fatty acid esters, Tween 20, Tween 80, Triton-X100, polyethylene glycol monooleyl ether, triethylene glycol monododecyl ether, octyl glucoside and nonanoyl methylglucamine.

Examples of anionic surfactants include acyl sarcosines, sodium alkyl sulfates, alkylbenzene sulfonates and fatty acid sodium salts having 7 to 22 carbon atoms. Specific examples include sodium dodecyl sulfate, sodium lauryl sulfate, sodium cholate, sodium deoxycholate and sodium taurodeoxycholate.

Examples of cationic surfactants include alkyl amine salts, acyl amine salts, quaternary ammonium salts and amine derivatives. Specific examples include benzalkonium chloride, acylaminoethyldiethylamine salts, N-alkyl polyalkyl polyamine salts, fatty acid polyethylene polyamides, cetyltrimethyl ammonium bromide, dodecyltrimethyl ammonium bromide, alkyl polyoxyethylene amines, N-alkylaminopropylamines and fatty acid triethanolamine esters.

Examples of amphoteric surfactants include dodecylbetaine, dodecyl dimethyl amine oxide, dimethylpalmityl ammoniopropane sulfonate, 3-[(3-cholamidopropyl)dimethylammonio]-1-propane sulfonate and N-tetradecyl-N,N-dimethyl-3-ammonio-1-propane sulfonate.

Alcohols added to lower viscosity of the oily component are composed of 2% by weight to 5% by weight alcohol. The alcohol may be selected from among C1 to C5 alcohols and mixtures thereof. Preferable examples of alcohols include methanol, ethanol, propanol, isopropanol, t-butanol, isobutanol and mixtures thereof. The alcohol is preferably ethanol.

Decomposition reaction conditions are preferably such that the temperature is controlled to about 20° C. to 40° C., preferably to 25° C. to 35° C., and more preferably to about 30° C., pH is adjusted to 6 to 9 and preferably to 6.5 to 8, and treatment is preferably carried out for about 12 hours to 72 hours while stirring. This treatment can be carried out using a closed reactor equipped for stirring, and namely, treatment is preferably carried out using a compact, dedicated device. Being able to reduce the size of the polychlorinated biphenyl decomposition reaction device makes it possible to carry out treatment work directly even at a storage location where trace PCBs are stored.

[Contaminant Detoxification Device]

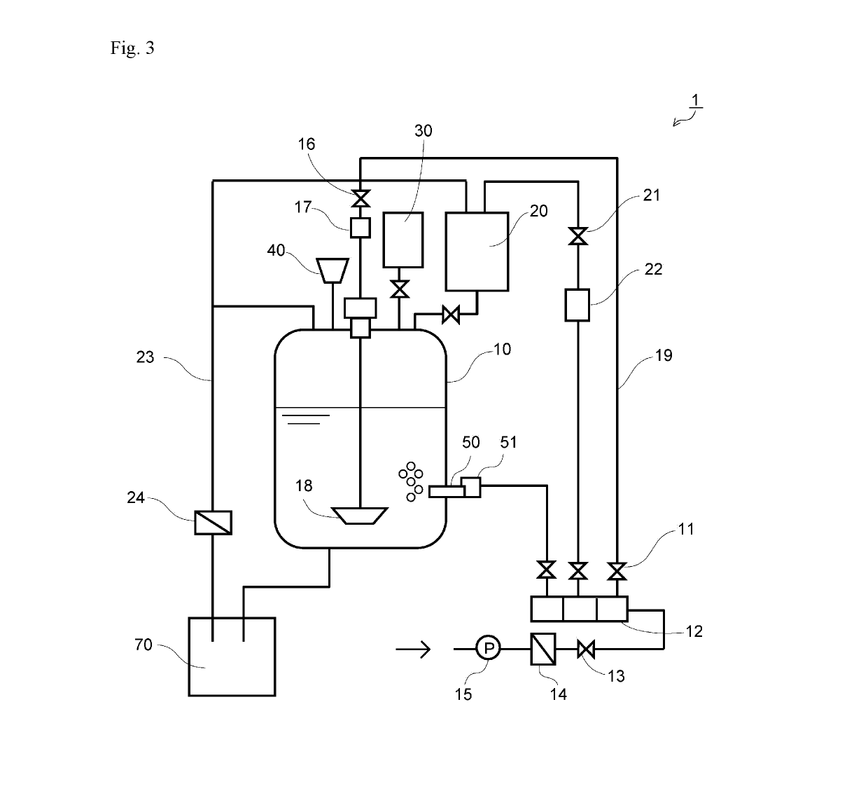

The following provides a description of a contaminant detoxification device of one embodiment of the present invention with reference to the drawings. In FIG. 3, a contaminant detoxification device 1 according to the present embodiment is provided with a stirring tank 10 provided with a stirrer 18 also serving as an aeration means, a buffer tank 20 in communication with the stirring tank for supplying an aqueous medium, a tank 30 for supplying the contaminant, an enzyme preparation feed port 40, and an air pipe 19 and exhaust pipe 23 for supplying thereto or discharging oxygen gas. In this embodiment, although a contaminant such as PCBs is supplied to the stirring tank from a dedicated tank, there are no particular limitations thereon, and the dedicated tank can also serve as a buffer tank. A microbubble generation unit composed of an ultrasonic vibration transmitter 50 and an ultrasonic vibrator 51 is further provided in the stirring tank.

Microbubbles can be contained in the aqueous medium by carrying out ultrasonic treatment on a mixture of the aqueous medium and oily component containing the contaminant introduced into the aforementioned stirring tank 10 while allowing oxygen to flow in from the aforementioned microbubble generation unit. Alternatively, a microbubble-containing aqueous medium and oily component containing the contaminant may be mixed after having preliminarily containing microbubbles in the aqueous medium present in the buffer tank 20. An ultrasonic microbubble generation device or a pressurized microbubble generation device can be used for the microbubble generation device used for this purpose.

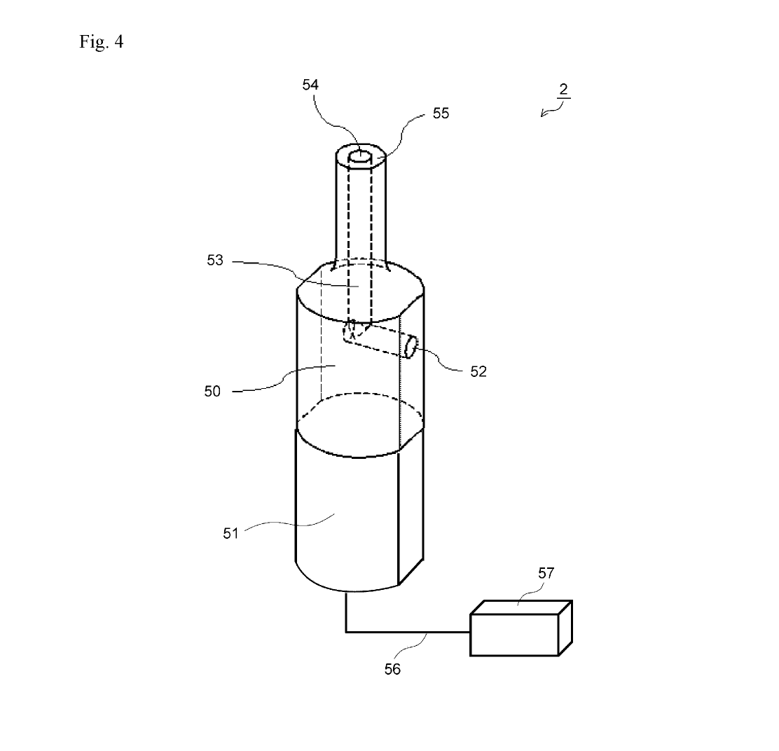

As long as the aforementioned microbubble generation unit is able to be arranged at a location in contact with the mixture of the aqueous medium and oily component in the aforementioned stirring tank, there are no particular limitations on the form thereof, but the ultrasonic microbubble generation device 2 shown in FIG. 4 can be used as a preferred embodiment. In addition, the supply of gas to liquid surrounding the vibrating body is such that gas can be supplied to liquid within a range over which vibrations from the aforementioned vibrating body 50 are effectively transmitted, and ensuring that as large a proportion of the supplied gas as possible contacts the vibrating body is preferable in terms of the efficiency at which microbubbles are formed. The form of the aforementioned gas supply is such that a gas flow path 53 that connects a gas supply port 52 and a gas release port 54 is preferably provided in the aforementioned vibrating body 50, gas is supplied to the gas supply port 52 from a compressor, and gas is released from the gas release port 54 located in the liquid in the aforementioned stirring tank. Vibrations of a prescribed frequency and amplitude can be imparted by arranging an ultrasonic wave radiation surface 55 of the aforementioned vibrating body in the liquid in the stirring tank and supplying an electrical signal to the vibrating body. Although there are no particular limitations on the shape of the aforementioned vibration transmission body 50, a known shape in the form of an amplitude magnifying horn commonly used to increase the amplitude of ultrasonic waves is preferable. The stepped cylindrical shape exemplified in FIG. 4 is a preferable shape of the aforementioned vibration transmission body 50. In FIG. 4, the ultrasonic vibrator 51 is connected to the end having the larger area, and the end having the smaller area serves as the aforementioned ultrasonic wave radiation surface 55 where vibrations generated with the aforementioned ultrasonic vibrator 51 are amplified. In addition, the aforementioned gas release port 54 is preferably provided on the aforementioned ultrasonic wave radiation surface 55 where vibrations are amplified. The aforementioned vibration transmission body 50 may be a structure composed of a single component, or may be a structure in which multiple components are connected by fastening together with screws, adhesion, welding or the like, provided it is capable of transmitting ultrasonic pressure vibrations.

Although there are no limitations on the material used for the aforementioned vibration transmission body 50, a known material used as an ultrasonic horn material is preferable, and examples thereof include titanium alloys, pure titanium, Ni—Cr steel, stainless steel, brass, Monel metal and tool steel.

There are no particular limitations on the ultrasonic vibrator 51 that composes the aforementioned ultrasonic microbubble generation unit, and is selected as appropriate from among known ultrasonic vibrators. The frequency and amplitude of ultrasonic waves generated by the aforementioned ultrasonic vibrator are controlled by signals of an arbitrary frequency and waveform generated in a vibration controller 57 in the form of electrical signals transmitted through a cable 56. The generation of microbubbles according to the present invention is such that, by imparting a certain minimum speed and certain minimum amplitude when causing gas bubbles present in a liquid to vibrate with ultrasonic waves, microbubbles are generated due to division of a portion of the gas-liquid interface of the gas bubbles or on the vibrator. Consequently, the higher the frequency and the greater the amplitude of the ultrasonic waves used, the higher the efficiency of microbubble generation is and the finer the generated gas bubbles can be. The frequency and amplitude of the ultrasonic waves used are suitably selected over a range of 10 μm or more and 10 kHz or more, respectively, corresponding to the purpose of microbubble generation. The frequency is preferably 15 kHz to 100 kHz, which is used by a large number of known ultrasonic vibrators, and more preferably covers a range of 20 kHz to 40 kHz that enables amplitude to be increased. In addition, ultrasonic waves may be applied continuously or may be applied in a burst mode in which ultrasonic waves are repeatedly generated and interrupted at a frequency equal to or less than the frequency of the ultrasonic waves applied.

[Pressurized Microbubble Generation Device]

Moreover, a device that generates microbubbles by reducing the pressure of water in which a gas containing a substrate has been dissolved under pressure and supplies the microbubbles to the aqueous medium in the buffer tank 20 can also be used as a microbubble generation device. Alternatively, any device may be used such as a device that causes a gas-liquid biphasic flow to collide with projections or collision bodies to shear the gas bubbles into microbubbles and supplies the microbubbles to a mixed liquid in the stirring tank 10.

### EXAMPLES

The following provides a detailed description of the contaminant detoxification method of the present invention by indicating examples and comparative examples relating to experimental methods relating to preparation of the oxygenase-containing composition and decomposition reaction along with those results. Furthermore, the present invention is not limited to these examples.

### Reference Example 1

### Culturing of PCB-Decomposing Bacteria and Preparation Formulation

A synthetic mineral salt medium (W medium) having the composition shown in Table 1 below was used to culture Comamonas testosteroni (C. testosteroni) strains YAZ2 and YU14-111.

TABLE 1

| Component | Content | |
|---|---|---|
| KH₂PO₄ | 1.7 | g/L |
| Na₂HPO₄ | 9.8 | g/L |
| (NH₄)₂SO₄ | 1.0 | g/L |
| MgSO₄•7H₂O | 0.1 | g/L |
| FeSO₄•7H₂O | 0.95 | mg/L |
| MgO | 10.75 | mg/L |
| CaCO₃ | 2.0 | mg/L |
| ZnSO₄•7H₂O | 1.44 | mg/L |
| CuSO₄•5H₂O | 0.25 | mg/L |
| CoSO₄•7H₂O | 0.28 | mg/L |
| H₃BO₃ | 0.06 | mg/L |
| conc. HCl | 51.3 | μL/L |

Culturing was first carried out using test tubes and flasks. Inoculums of strain YAZ2 and strain YU14-111 (YAZ Library, Applizyme, Inc.) were inoculated into 10 volumes of W medium containing biphenyl added to a final concentration of 0.1% followed by culturing at a shaking speed of 120 rpm and temperature of 30° C. After confirming that the bacteria had grown sufficiently, the entire amount was inoculated into 10 volumes of W medium containing 0.1% biphenyl and re-cultured. A pre-culture broth containing an adequate amount of bacteria for final culturing was obtained by further repeating the same procedure one more time. A jar fermenter (Model BMS-C, Able Corp.) having a volume of 5 L was used for final culturing. The entire amount of the aforementioned pre-culture broth was added to the jar fermenter and 3 L of culture broth were cultured at an aeration rate of 4 L/min, stirring speed of 600 rpm and temperature of 30° C. Biphenyl used for the carbon source was additionally added as appropriate based on the amount of oxygen consumed by the cultured microbial cells (dissolved oxygen concentration in the culture broth). After recovering microbial cells from the culture broth by a centrifugation procedure, the cells were promptly frozen with liquid nitrogen to prevent deactivation of the expressed biphenyl dioxygenase and then immediately stored in a freezer at −80° C.

### Reference Example 2

### Confirmation of Biphenyl Dioxygenase Gene Carried by Bacteria

Several locations of highly preserved regions of BphA1 (biphenyl-2,3-dioxygenase a subunit) of known PCB-decomposing bacteria were selected based on a comparison of the amino acid sequences thereof (such as Asn-Gln/Ser-Cys-Arg/Ser-His-Arg-Gly-Met (SEQ ID NO: 1) or Glu-Gln-Asp-Asp-Gly/Thr-Glu-Asn (SEQ ID NO: 2)), and then used to create degenerate primers.

Next, bacteria of Comamonas testosteroni (C. testosteroni) strains YAZ2 and YU14-111 were suspended in a suitable amount of TE buffer (10 mM Tris-HCl, 1 mM EDTA, pH 8.0) and subjected to heat treatment, and the resulting extract containing genomic DNA was used as a template. PCR was then carried out using the resulting degenerate primers under reaction conditions consisting of 3 minutes at 94° C., 30 cycles of 30 seconds at 94° C., 30 seconds at 58° C. to 60° C. and 1 minute at 72° C., and finally 2 minutes at 72° C. In addition, a similar reaction was carried out using a thermal extract containing the genome of Escherichia coli strain K-12 as a negative control that does not carry the BphA1 gene.

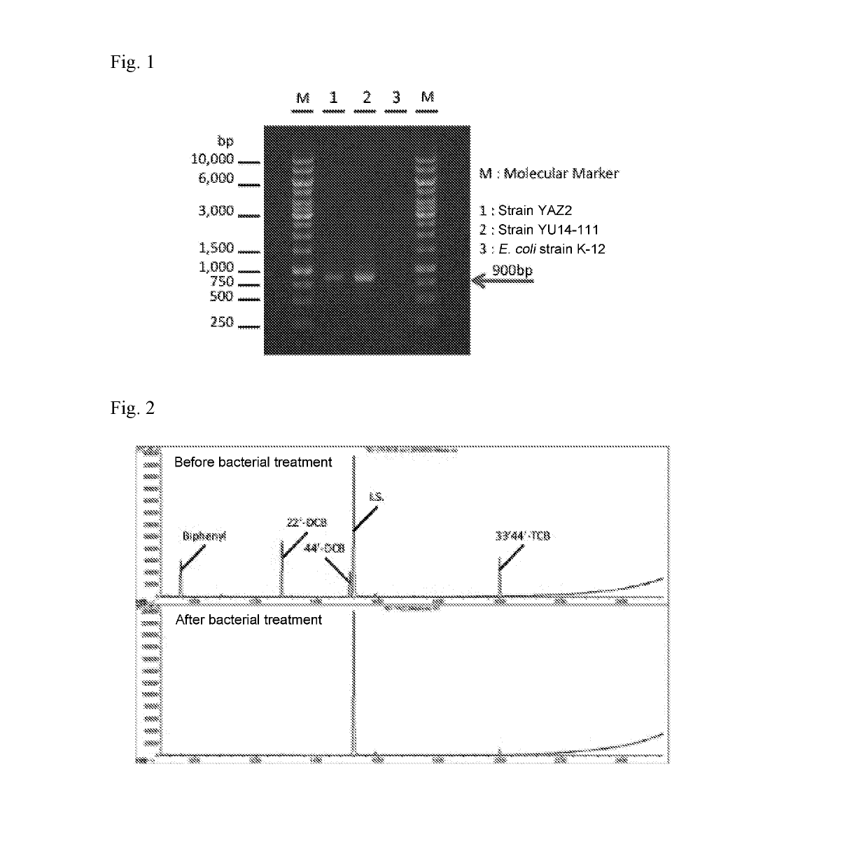

As a result, as shown in FIG. 1, the predicted approximately 900 bp BphaA1 fragment amplification products were detected for both strains YAZ2 and YU14-111, thereby confirming that these strains carry the BphA1 gene. This test was repeated three times.

### Example 1

### PCB Decomposition Test Using Bacteria Expressing Biphenyl Dioxygenase Gene

A bacterial preparation of C. testosteroni strain YAZ2 expressing the biphenyl dioxygenase gene was prepared in the same manner as the aforementioned Reference Example 1 and this bacterial preparation was adjusted to a turbidity at a wavelength of 660 nm of 10 or 60 (wet weight of approximately 15 mg or 90 mg) followed by contacting and reacting over the course of 24 hours while warming at 30° C. with a mixed PCB solution obtained by mixing biphenyl and PCB isomer standards in the form of 2,2′-dichlorobiphenyl, 4,4′-dichlorobiphenyl and 3,3′,4,4′-tetrachlorobiphenyl at concentrations of 0.5 ppm each. This test was repeated twice.

Recovery of residual PCBs following contact and reaction with the bacterial preparation was carried out by liquid-liquid extraction using ethyl acetate. More specifically, a procedure consisting of adding two volumes of ethyl acetate to a sample solution containing anthracene as internal standard and stirring well followed by recovering the upper organic solvent layer by a centrifugation procedure was repeated twice. The organic solvent layer was suitably diluted after dehydrating with anhydrous sodium sulfate and then applied to a gas chromatograph mass spectrometer (5957GC/MSD, Agilent Technologies Inc.).

The temperature program used for gas chromatography consisted of raising the temperature from an initial temperature of 80° C. to 130° C. at a heating rate of 20° C./min followed by continuing to raise the temperature to 300° C. at a rate of 8° C./min. A non-polar capillary column (HP-5 ms, 0.25 mm×15 m, 0.25 μm, Agilent Technologies Inc.) was used for the analysis column.

Total ion chromatogram analysis charts (to be referred to as TIC charts) from the gas chromatograph mass spectrometer of a test sample, obtained by contacting and reacting the bacterial preparation and PCBs, and a mixture of PCB isomer standards are shown in FIG. 2.

A comparison of the two TIC charts indicates that peaks corresponding to biphenyl as well as PCB isomers in the form of 2,2′-dichlorobiphenyl, 4,4′-dichlorobiphenyl and 3,3′,4,4′-tetrachlorobiphenyl had disappeared in the test sample.

Namely, bacteria expressing the biphenyl dioxygenase gene were shown to decompose biphenyl and PCB isomers. This result suggested that the bacterial preparation contains a PCB-decomposing enzyme in the form of biphenyl dioxygenase.

### Example 2

### PCB Decomposition Test Using Bacteria in the Presence of Oxygen Microbubbles

Oxygen microbubbles were continuously filled into chilled 20 mM sodium phosphate buffer (pH 7.5) for 10 minutes using a device in which a hollow horn (outlet inner diameter/outer diameter: 2.6 mm/6.0 mm) that allows gas to pass therethrough was attached to an ultrasonic homogenizer (UH-50, SMT Co., Ltd.) as shown in FIG. 4. Microbubbles having a diameter of 20 μm or less were generated from this device, and the dissolved oxygen concentration in the 20 mM sodium phosphate buffer was increased to about 28 ppm by bubbling for 10 minutes.

The surfactant Triton X-100 to a final concentration of 0.01% or 0.005%, a bacterial preparation expressing biphenyl dioxygenase at a turbidity at a wavelength of 660 nm of 10 or 60 (wet weight of approximately 15 mg or 90 mg), and PCB-contaminated waste oil or a commercially available PCB product in the form of Kanechlor KC-300 (GL Sciences Inc.) at a concentration of 10 ppm or 100 ppm were added per 0.5 mL of the microbubble-filled solution to obtain a PCB decomposition reaction solution followed by agitating by inverting while warming at 30° C. At this time, the mixing ratio of PCB-containing oil to 20 mM sodium phosphate buffer was a volume ratio of about 99:1. The test was repeated three times.

Recovery of residual PCBs in the decomposition reaction solution was carried out by liquid-liquid extraction using ethyl acetate in the same manner as Example 1. Analysis by gas chromatograph mass spectrometer and temperature programming for gas chromatography were carried out with the 5975GC/MDS manufactured by Agilent Technologies Inc., and the temperature was raised from an initial temperature of 80° C. to 130° C. at a heating rate of 20° C./min followed by continuing to raise the temperature to 300° C. at a rate of 8° C./min in the same manner as Example 1. A non-polar capillary column (HP-5 ms, 0.25 mm×15 m, 0.25 μm, Agilent Technologies Inc.) was used for the analysis column.

Quantitative analysis was carried out using the internal standard method and analysis at each fixed point was repeated three times in all cases followed by determination of the average and standard deviation of measured values.

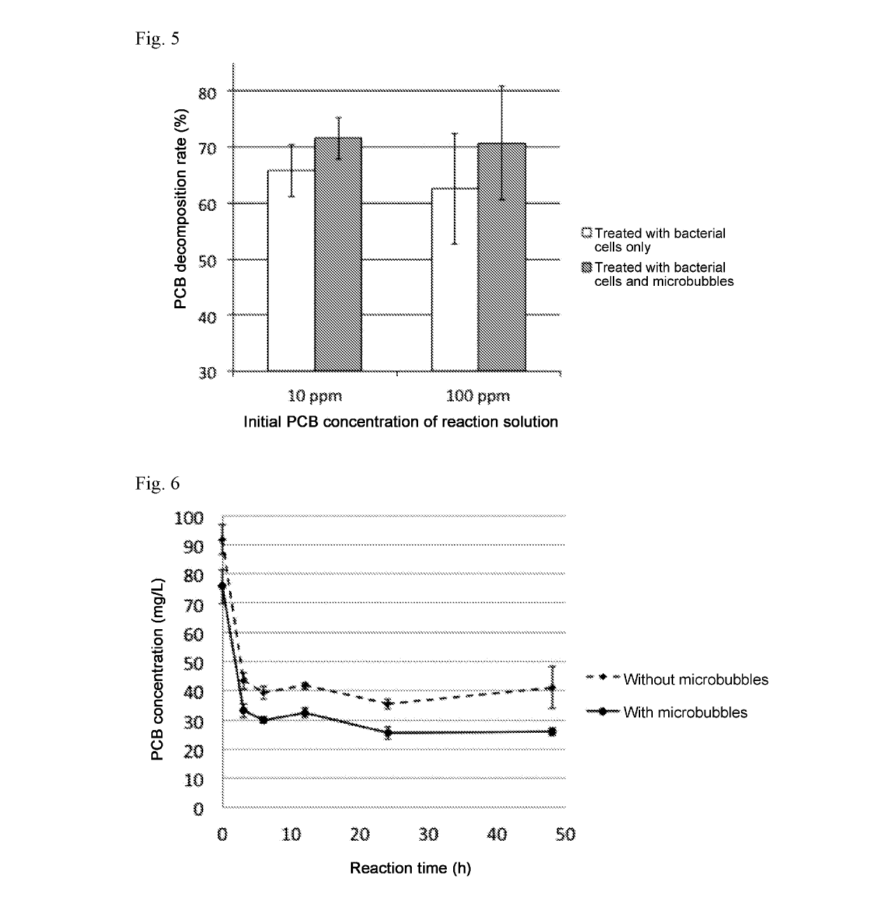

A biphenyl dioxygenase-expressing bacterial preparation consisting of C. testosteroni strain YU14-111 and PCB-contaminated waste oil (initial PCB concentration: 10 ppm and 100 ppm) were contacted and reacted in 20 mM sodium phosphate buffer in the presence of a high dissolved oxygen concentration (initial concentration: approx. 28 ppm) obtained by filling with oxygen microbubbles, and results for PCB decomposition rates after 24 hours are shown in FIG. 5.

In the decomposition reaction in which the initial PCB concentration was 10 ppm, in contrast to the decomposition rate for a control to which microbubbles were not added being 65.8±4.7%, decomposition rate rose to 71.6±3.7% with the addition of microbubbles. Similarly, in the case of an initial PCB concentration of 100 ppm, in contrast to the control decomposition rate being 62.6±9.9%, the decomposition rate in the presence of oxygen microbubbles was confirmed to increase to 70.7±10.1%.

On the basis of the above results, in comparison with a reaction at ordinary oxygen partial pressure, filling with oxygen microbubbles was determined to improve the efficiency of PCB decomposition by the C. testosteroni strain YU14-111 preparation by about 5% to 8%.

### Example 3

### Time-Based Changes in PCB Decomposition Rate in Presence of Oxygen Microbubbles

A C. testosteroni strain YAZ2 bacterial preparation highly expressing biphenyl dioxygenase and a commercially available PCB product in the form of Kanechlor KC-300 (initial concentration: 100 ppm) were allowed to react in 20 mM sodium phosphate buffer in the presence of a high dissolved oxygen concentration (initial concentration: approx. 28 ppm) obtained by filling with oxygen microbubbles using the same method as Example 1, followed by following time-based changes in residual PCB concentration for 48 hours, and those results are shown in FIG. 6.

In the case of filling with oxygen microbubbles, a high level of decomposition activity was demonstrated immediately after the start of the reaction in comparison with the case of not filling with oxygen microbubbles, and the difference in decomposition rates between the two was 16.1% (residual PCB concentration: 91.7±5.0 ppm without microbubble filling in contrast to 75.6±5.9 ppm with microbubble filling). The case of filling with oxygen microbubbles subsequently continued to demonstrate higher PCB decomposition rates at all measurement times, and after reacting for 48 hours in particular, the residual PCB concentration in the case of filling with oxygen microbubbles was 25.9±1.2 ppm while that in the case of not filling with oxygen microbubbles was 41.0±7.1 ppm, thus demonstrating a difference of 15.1 ppm. The average difference in residual PCB concentrations between the two cases for all measurement times was 11.6 ppm, and filling with oxygen microbubbles was confirmed to improve decomposition rate by an average of about 12%.

### Reference Example 3

### Construction of Expression Plasmid for Biphenyl Dioxygenase of Burkholderia Xenovorans Strain LB400

Biphenyl dioxygenase activity includes biphenyl-2,3-dioxygenase (to be referred to as 2,3-dioxygenase) activity and biphenyl-3,4-dioxygenase (to be referred to as 3,4-dioxygenase) activity. As a result of investigating the biphenyl dioxygenase of more than 200 strains of environmental microorganisms acquired in Japan thus far, all enzymes demonstrated 2,3-dioxygenase activity. Accordingly, the inventors of the present invention considered the acquisition of enzymes having 3,4-dioxygenase activity to be important for further improving PCB decomposition rate.

Plasmids were then produced using gene recombination for the purpose of acquiring enzymes having 3,4-dioxygenase activity for the reasons described above. A 2120 bp (SEQ ID NO: 3) or 1,600 bp (SEQ ID NO: 6) DNA sequence containing the BphA1A2 or BphA3A4 gene of Burkholderia xenovorans strain LB400 (to be referred to as strain LB400) was used for the gene serving as the motif. PCR was carried out under reaction conditions consisting of 3 minutes at 94° C., 28 cycles of 30 seconds at 94° C., 30 seconds at 60° C. to and 2 minutes at 68° C., and finally 3 minutes at 68° C. by combining primers 1 and 2 or primers 3 and 4 described below and using as templates plasmid pUC57-bphA1A2(LB400) or pUC57-bphA3A4(LB400), obtained by using artificial genes produced by organic chemical synthesis for these DNA sequences and inserting them each into a pUC57 cloning vector (Thermo Fisher Scientific Inc.).

PrimeSTAR HS DNA Polymerase (Takara Bio Inc.) is preferably used for the DNA polymerase required during PCR. The reason for this is that high-quality plasmids can be constructed by inhibiting the occurrence of erroneous gene substitutions that can occur during the PCR reaction.

Each of the sequences of the previously described primers 1 to 4 is as indicated below.

Primer 1:
5′-ATGCATTCTAGATATTTTTTCCGCCCTGCCAAG-3′
(underline: restrictase XbaI recognition sequence,
SEQ ID NO: 9)
Primer 2: 5′-ATGCATCCATGGCGTGCTGGGCTAGAAGAACAT-3′
(underline: restrictase NcoI recognition sequence,
SEQ ID NO: 10)
Primer 3: 5′-ATGCATCCATGGCCCAGGCGATTTAACCCTTTTA-3′
(underline: restrictase NcoI recognition sequence,
SEQ ID NO: 11)
Primer 4: 5′-ATGCATCATATGCGCATCAATTCGGTTTGGC-3′
(underline: restrictase NdeI recognition sequence,
SEQ ID NO: 12)

After respectively cleaving the DNA fragments containing BpHA1A2 or BphA3A4 gene of strain LB400 obtained in the aforementioned PCR with XbaI and NcoI or NcoI and NdeI, the fragments were purified by gel extraction and respectively inserted into the XbaI-NcoI or NcoI-NdeI restriction site of plasmid vector pET-15b (Novagen Inc.).

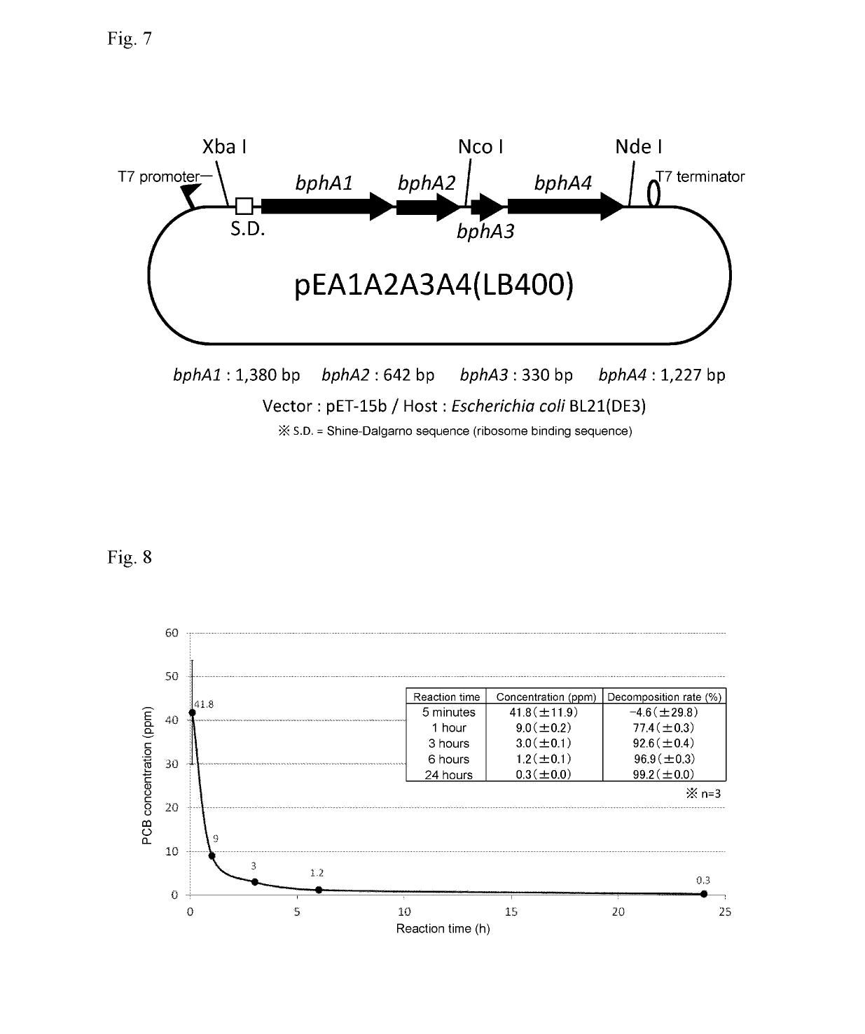

After respectively producing pET-15b-bphA1A2(LB400) and pET-15b-bphA3A4(LB400) in the manner previously described and checking each inserted DNA sequence for the absence of erroneous gene substitutions that can occur in the PCR reaction, the NcoI-NdeI fragment containing bphA3A4(LB400) was cut out and inserted at the NcoI-NdeI site downstream of pET-15b-bphA1A2 to ultimately obtain pEA1A2A3A4(LB400), an expression plasmid for BphA1A2A3A4 of strain LB400 (FIG. 7).

### Example 4

### Test Using Compact PCB Decomposition Device Equipped with Oxygen Microbubble Generation Mechanism

The efficiencies of decomposition of various PCB isomers were verified with a compact decomposition device (FIG. 3) using microbial catalysts obtained by compounding an E. coli strain expressing BphA1A2A3A4(LB400) and wild type Comamonas testosteroni strain YU14-111, which express two types of dioxygenase having different PCB isomer decomposition characteristics.

E. coli strain BL21(DE3) (Novagen Inc.) transformed with plasmid pEA1A2A3A4(LB400) produced in Reference Example 3 was cultured to an OD₆₆₀ of 4.0 to 5.0, and preferably 5.0, at a temperature of 30° C. using 2×YT medium containing 100 μg/ml of ampicillin, followed by harvesting 90 minutes after adding IPTG to a final concentration of 0.2 mM. After washing the harvested microbial cells with buffer, the cells were used after re-suspending in the same buffer as that used during washing. By contrast, preparation of wild type Comamonas testosteroni strain YU14-111 was carried out by weighing out the required amount of a preparation produced in the same manner as the method described in Japanese Unexamined Patent Publication No. 2013-179890 and washing with the same buffer as described above followed by using after re-suspending in the same buffer.

In the present study, a compact decomposition device equipped with a mechanism capable of generating microbubbles using the pressurization method was used for the compact decomposition device capable of generating oxygen microbubbles. The following provides a description of the reaction procedure.

First, sodium phosphate buffer preliminarily filled with oxygen microbubbles using the pressurization method and having a dissolved oxygen concentration of 20 ppm or more and preferably 28 ppm or more was introduced into the PCB decomposition reaction tank equipped in the aforementioned compact decomposition device. Next, a preparation obtained by compounding E. coli microbial cells expressing BphA1A2A3A4(LB400) and wild type Comamonas testosteroni strain YU14-111 cells at an OD₆₆₀ turbidity ratio of 16:4 was added. Continuing, PCB-contaminated insulating oil (final PCB concentration: 40 ppm) and surfactant Triton X-100 having a final concentration of 0.001% to 0.01%, and preferably 0.005%, were added followed by carrying out a decomposition reaction after bringing the final volume of the reaction liquid to 1 L. The temperature in the reaction tank during the reaction was maintained at 30° C.±2° C. The concentration of dissolved oxygen during the reaction was adjusted so as to maintain a concentration of 20 ppm to 40 ppm, and preferably 28 ppm or more, by continuously or intermittently supplying oxygen gas so as to be added to the reaction tank for which partial pressure had been increased in advance. Oxygen was added by aerating with oxygen gas from the bottom of the reaction tank or using a sparger made of PTFE in which the oxygen microbubble filling port provided on the side at the bottom of the reaction tank had been modified to have as large a number as possible of pores having a diameter of 1 μm or less and penetrating therethrough. The reaction liquid was stirred to carry out the optimum reaction, or in other words, to optimally carry out the dispersion and contact reaction of PCBs and the compound microbial catalyst in the reaction liquid. A stirring force equivalent to 40 rpm was imparted while using the physical stirring force of a stirrer or the ascent force of oxygen aeration or oxygen microbubbles.

A portion of the reaction liquid was respectively sampled at 5 minutes, 1 hour, 3 hours, 6 hours and 24 hours after initiating contact between PCBs and the compound microbial catalyst, and the results of measuring time-based changes in the amount of residual PCBs using GC-MS in the same manner as Example 1 are shown in FIG. 8 and Table 2.

TABLE 2

| Reaction time | | PCB concentration (ppm) | PCB decomposition rate (%) |
|---|---|---|---|
| 5 | minutes | 41.8 (±11.9) | −4.6 (±29.8) |
| 1 | hour | 9.0 (±0.2) | 77.4 (±0.3) |
| 3 | hours | 3.0 (±0.1) | 92.6 (±0.4) |
| 6 | hours | 1.2 (±0.1) | 96.9 (±0.3) |
| 24 | hours | 0.3 (±0.0) | 99.2 (±0.0) |

As a result of measurement, the initially added 40 ppm of PCBs rapidly decreased to 9.0±0.2 ppm 1 hour after the start of the reaction, and further decreased to 3.0±0.1 ppm at 3 hours after the start of the reaction. When expressed in terms of decomposition rate, an extremely high decomposition rate of 92.6±0.4% was demonstrated. Moreover, decomposition proceeded down to 1.2±0.1 ppm (decomposition rate: 96.9±0.3%) 6 hours after the start of the reaction, and after 24 hours, PCBs were stably decomposed to 0.3±0.0 ppm (99.2±0.0%), thereby demonstrating extremely high activity and highly efficient decomposition performance that is below the regulation criterion established by the Ministry of the Environment of 0.5 ppm. The aforementioned analysis was repeated three time (n=3).

### BRIEF DESCRIPTION OF THE REFERENCE SYMBOLS

1 Contaminant detoxification device
2 Microbubble generation device
10 Stirring tank
11 Switch valve
12 Regulator
13 Main valve
14 Filter regulator
15 Air pump (compressor)
16 Check valve
17 Rotary joint
18 Stirrer
19 Air pipe
20 Buffer tank
30 PCB tank
40 Microbial preparation feed port
50 Vibration transmission body
51 Ultrasonic vibrator
52 Gas supply port
53 Gas flow path
54 Gas release port
55 Ultrasonic wave radiation surface
56 Cable
57 Vibration controller

1. A method for activating an oxygenase-containing composition, characterized by dissolving or dispersing a composition containing oxygenase in an aqueous medium containing an amount of oxygen that exceeds the saturated dissolved oxygen concentration in an atmospheric environment at normal temperature and normal pressure.

2. A method for decomposing or detoxifying a contaminant, comprising: stirring a mixture of an oxygenase-containing composition and an oily component containing a contaminant in an aqueous medium containing an amount of oxygen that exceeds the saturated dissolved oxygen concentration.

3. The method according to claim 1 or 2, comprising supplying microbubbles to the aqueous medium.

4. The method according to claim 3, wherein the microbubbles are generated by carrying out ultrasonic treatment while allowing oxygen to flow to the aqueous medium and/or a mixture of the aqueous medium and the oily component.

5. The method according to claim 3, wherein the microbubbles are generated by subjecting the aqueous medium and/or the mixture of aqueous medium and oily component to pressurization treatment.

6. The method according to claim 2, wherein the contaminant is composed of monocyclic or polycyclic aromatic compounds including toluene, benzene, dioxins and/or polychlorinated biphenyls.

7. The method according to claim 1, wherein the oxygenase-containing composition is a microbial preparation obtained by expressing aromatic ring-hydroxylating oxygenase within microbial cells.

8. The method according to claim 2, wherein the mixture optionally contains a surfactant or alcohol, and is an emulsion of the aqueous medium and the oily component.

9. A contaminant detoxification device, provided with:
a stirring tank provided with an aeration means, stirring means and/or temperature control means,
a sample introduction unit that introduces an aqueous medium and/or oily component containing a contaminant, the sample introduction unit being in communication with the stirring tank, and
a microbubble generation unit that supplies microbubbles to a sample in the stirring tank.

10. The device according to claim 9, wherein the microbubble generation unit is an ultrasonic microbubble generation device provided in the stirring tank.

11. The device according to claim 9, wherein the microbubble generation unit is a pressurized microbubble generation device provided in the sample introduction unit.

12. The device according to any of claims 9 to 11 for use in a method for activating an oxygenase-containing composition, characterized by dissolving or dispersing a composition containing oxygenase in an aqueous medium containing an amount of oxygen that exceeds the saturated dissolved oxygen concentration in an atmospheric environment at normal temperature and normal pressure.

13. A method for decomposing or detoxifying a contaminant, comprising:
mixing an oxygenase-containing composition and an oily component containing a contaminant in an aqueous medium containing oxygen in an amount that exceeds the saturated dissolved oxygen concentration, at least a portion of the oxygen being present as microbubbles, and
aerating and stirring the mixture of the aqueous medium and the oily component.


SEQUENCE LISTING (12 sequences)

SEQ ID NO: 1 - protein, 8 aa, Artificial Sequence
  primer sequence
  MISC_FEATURE (2)..(2): Xaa means Gln or Ser
  MISC_FEATURE (4)..(4): Xaa means Arg or Ser
NXCXHRGM

SEQ ID NO: 2 - protein, 7 aa, Artificial Sequence
  primer sequence
  MISC_FEATURE (5)..(5): Xaa means Gly or Thr
EQDDXEN

SEQ ID NO: 3 - DNA, 2120 bp, Burkholderia sp. LB400
  CDS (47)..(1426): bphA1
  CDS (1469)..(2110): bphA2
tattttttccgccctgccaagggcatttcaacggagacgttaaatcatgagttcagcaatcaaagaagtgcagggagcccctgtgaagtgggttaccaattggacgccggaggcgatccgggggttggtcgatcaggaaaaagggctgcttgatccacgcatctacgccgatcagagtctttatgagctggagcttgagcgggtttttggtcgctcttggctgttacttgggcacgagagtcatgtgcctgaaaccggggacttcctggccacttacatgggcgaagatccggtggttatggtgcgacagaaagacaagagcatcaaggtgttcctgaaccagtgccggcaccgcggcatgcgtatctgccgctcggacgccggcaacgccaaggctttcacctgcagctatcacggctgggcctacgacatcgccggcaagctggtgaacgtgccgttcgagaaggaagccttttgcgacaagaaagaaggcgactgcggctttgacaaggccgaatggggcccgctccaggcacgcgtggcaacctacaagggcctggtctttgccaactgggatgtgcaggcgccagacctggagacctacctcggtgacgcccgcccctatatggacgtcatgctggatcgcacgccggccgggactgtggccatcggcggcatgcagaagtgggtgattccgtgcaactggaagtttgccgccgagcagttctgcagtgacatgtaccacgccggcaccacgacgcacctgtccggcatcctggcgggcattccgccggaaatggacctctcccaggcgcagatacccaccaagggcaatcagttccgggccgcttggggcgggcacggctcgggctggtatgtcgacgagccgggctcactcctggcggtgatgggccccaaggtcacccagtactggaccgagggtccggctgccgagcttgcggaacagcgcctggggcacaccggcatgccggttcgacgcatggtcggccagcacatgacgatcttcccgacctgttcattcctgcccaccttcaacaacatccggatctggcacccgcgtggtcccaatgaaatcgaggtgtgggccttcaccctggtcgatgccgacgccccggcggagatcaaggaagaatatcgccggcacaacatccgcaacttctccgcaggcggcgtgtttgagcaggacgatggcgagaactgggtggagatccagaaggggctacgtgggtacaaggccaagagccagccgctcaatgcccagatgggcctgggtcggtcgcagaccggtcaccctgattttcctggcaacgtcggctacgtctacgccgaagaagcggcgcggggtatgtatcaccactggatgcgcatgatgtccgagcccagctgggccacgctcaagccctgatcaagacgcaatcgttagatctgtcaaccggaagaattcaacatggtgggctggacgtgcatgtgcagacggcgcgccgaggttccgtcccctgatatttacttggagataactgttatgacaaatccatccccgcattttttcaaaacatttgaatggccaagcaaggcggctggccttgagttgcagaacgagatcgagcagttctactaccgcgaagcgcagttgcttgaccaccgggcctacgaggcctggtttgccctgctggacaaagatatccactacttcatgccgctgcgcaccaatcgcatgatccgggagggcgagctggaatattccggcgaccaggatttagcccatttcgatgaaacccatgaaaccatgtacgggcgcatccgcaaggtgacctcggacgtgggctgggcggagaacccgccttcccgcacgcgccacctggtctccaacgtcatcgtcaaggagacggccacgccggataccttcgaggtcaattccgcattcatcctgtaccgcaatcggcttgagcgccaggtcgacatcttcgcgggcgaacgccgggacgtgctgcgccgcgccgacaacaaccttggtttcagcatcgccaagcgcaccatcctgctcgacgccagtaccttgctgtcgaacaacctgagcatgttcttctagcccagcacgc

SEQ ID NO: 4 - protein, 459 aa, Burkholderia sp. LB400
MSSAIKEVQGAPVKWVTNWTPEAIRGLVDQEKGLLDPRIYADQSLYELELERVFGRSWLLLGHESHVPETGDFLATYMGEDPVVMVRQKDKSIKVFLNQCRHRGMRICRSDAGNAKAFTCSYHGWAYDIAGKLVNVPFEKEAFCDKKEGDCGFDKAEWGPLQARVATYKGLVFANWDVQAPDLETYLGDARPYMDVMLDRTPAGTVAIGGMQKWVIPCNWKFAAEQFCSDMYHAGTTTHLSGILAGIPPEMDLSQAQIPTKGNQFRAAWGGHGSGWYVDEPGSLLAVMGPKVTQYWTEGPAAELAEQRLGHTGMPVRRMVGQHMTIFPTCSFLPTFNNIRIWHPRGPNEIEVWAFTLVDADAPAEIKEEYRRHNIRNFSAGGVFEQDDGENWVEIQKGLRGYKAKSQPLNAQMGLGRSQTGHPDFPGNVGYVYAEEAARGMYHHWMRMMSEPSWATLKP

SEQ ID NO: 5 - protein, 213 aa, Burkholderia sp. LB400
MVGWTCMCRRRAEVPSPDIYLEITVMTNPSPHFFKTFEWPSKAAGLELQNEIEQFYYREAQLLDHRAYEAWFALLDKDIHYFMPLRTNRMIREGELEYSGDQDLAHFDETHETMYGRIRKVTSDVGWAENPPSRTRHLVSNVIVKETATPDTFEVNSAFILYRNRLERQVDIFAGERRDVLRRADNNLGFSIAKRTILLDASTLLSNNLSMFF

SEQ ID NO: 6 - DNA, 1600 bp, Burkholderia sp. LB400
  CDS (42)..(371): bphA3
cccaggcgatttaacccttttaactaattacaagaagcgttatgaaatttaccagagtttgtgatcgaagagatgtgcccgaaggcgaagccctgaaggtcgaaagtggaggcacctccgtcgcgattttcaatgtggatggcgagctgttcgcaacacaggaccgctgcacccacggcgactggtccctgtccgatggcggctatcttgaaggtgacgtggtggaatgctcactgcacatggggaagttttgcgttcgcacgggcaaggtcaaatcaccgccgccctgtgaggcactgaagatatttccgatccgcatcgaagacaatgacgtgctggtcgacttcgaagccgggtatctggcgccatgatcgacaccatcgccatcatcggcgccggcctggccggttcgacggctgcgcgcgcactgcgcgcccagggatacgaggggcgcatccacctgctcggggatgagtcgcatcaggcctatgaccggaccacgctgtccaagacggtgctggcgggcgagcagcccgagccgcctgcaatcctggacagcgcctggtacgcatcggcccatgtggatgtccagctcgggcgacgggtgagttgcctggatctggccaaccgccagattcagtttgaatcgggcgccccgctggcctacgaccggctgctgctggccaccggcgcgcgcgcccggcgcatggcgattcggggtggcgacctggcaggcatccataccttgcgagacctcgccgacagccaggcgctgcggcaggcgctgcaaccgggccagtcgctggtcatcgtcggcggaggcctgatcggttgcgaggtggcgaccaccgcccgcaagctgagtgtccatgtcacgattctggaagccggcgacgagttgctggtgcgcgtgctgggtcaccggaccggggcatggtgtcgggccgaactggaacgcatgggtgtccgcgtggagcgcaatgcacaggccgcgcgcttcgaaggccaggggcaggtgcgcgccgtgatctgcgccgacgggcgccgggtgcccgccgatgtggtcttggtcagcattggcgccgagccggcggacgagctggcccgtgccgctggcatcgcctgcgcgcgcggcgtgctggtcgacgccaccggcgccacctcgtgtccagaggtgttcgccgccggtgacgtcgccgcctggccgctgcgtcaagggggccagcgctcgctggagacctacctgaacagccagatggaggccgaaatcgcggccagcgccatgttgagtcagcccgtgccggcgccccaggtgccgacctcgtggacggagattgcaggccaccgcatccagatgattggcgatgccgaagggcccggcgagatcgtcgtacgcggcgacgcccagagcggccagccaatcgtgttgctcaggctgcttgatggctgcgtcgaggccgcgacggcgatcaatgccaccagggaattttctgtggcgacccgactggtcggcacccgggtttctgtttccgccgagcaactgcaggacgtcggctcgaacctgcgggatttactcaaagccaaaccgaattgatgcgca

SEQ ID NO: 7 - protein, 109 aa, Burkholderia sp. LB400
MKFTRVCDRRDVPEGEALKVESGGTSVAIFNVDGELFATQDRCTHGDWSLSDGGYLEGDVVECSLHMGKFCVRTGKVKSPPPCEALKIFPIRIEDNDVLVDFEAGYLAP

SEQ ID NO: 8 - protein, 408 aa, Burkholderia sp. LB400
  MISC_FEATURE: BphA4
MIDTIAIIGAGLAGSTAARALRAQGYEGRIHLLGDESHQAYDRTTLSKTVLAGEQPEPPAILDSAWYASAHVDVQLGRRVSCLDLANRQIQFESGAPLAYDRLLLATGARARRMAIRGGDLAGIHTLRDLADSQALRQALQPGQSLVIVGGGLIGCEVATTARKLSVHVTILEAGDELLVRVLGHRTGAWCRAELERMGVRVERNAQAARFEGQGQVRAVICADGRRVPADVVLVSIGAEPADELARAAGIACARGVLVDATGATSCPEVFAAGDVAAWPLRQGGQRSLETYLNSQMEAEIAASAMLSQPVPAPQVPTSWTEIAGHRIQMIGDAEGPGEIVVRGDAQSGQPIVLLRLLDGCVEAATAINATREFSVATRLVGTRVSVSAEQLQDVGSNLRDLLKAKPN

SEQ ID NO: 9 - DNA, 33 bp, Artificial Sequence
  PCR primer 1 containing XbaI site
atgcattctagatattttttccgccctgccaag

SEQ ID NO: 10 - DNA, 33 bp, Artificial Sequence
  PCR primer 2 containing NcoI site
atgcatccatggcgtgctgggctagaagaacat

SEQ ID NO: 11 - DNA, 34 bp, Artificial Sequence
  PCR primer 3 containing NcoI site
atgcatccatggcccaggcgatttaaccctttta

SEQ ID NO: 12 - DNA, 31 bp, Artificial Sequence
  PCR primer 4 containing NdeI site
atgcatcatatgcgcatcaattcggtttggc